(19) United States
(12) Patent Application Publication
(10) Pub. No.: US 2026/0043785 A1
(43) Pub. Date: Feb. 12, 2026

(54) METHOD FOR DETERMINING MIGRATION PATH OF ORE-FORMING HYDROTHERMAL FLUID BASED ON RUTILE

(71) Applicants: Tibet Julong Copper Co., Ltd., Lhasa (CN); CHINA UNIVERSITY OF GEOSCIENCES (WUHAN), Wuhan (CN)

(72) Inventors: Xian CHE, Lhasa (CN); Xingkai HUANG, Lhasa (CN); Gen CHEN, Lhasa (CN); Xin CHEN, Wuhan (CN); Xiaofang DOU, Beijing (CN); Deng PAN, Lhasa (CN); Shunbao GAO, Wuhan (CN); Nan CHAO, Wuhan (CN); Zizhuo WU, Wuhan (CN)

(73) Assignees: Tibet Julong Copper Co., Ltd., Lhasa (CN); CHINA UNIVERSITY OF GEOSCIENCES (WUHAN), Wuhan (CN)

(21) Appl. No.: 19/364,115

(22) Filed: Oct. 21, 2025

(30) Foreign Application Priority Data
Oct. 24, 2024 (CN) 202411492391.3

(51) Int. Cl. G01N 33/24; H01J 49/16

(52) U.S. Cl. CPC G01N 33/24; H01J 49/164

(57) ABSTRACT

A method for determining a migration path of an ore-forming hydrothermal fluid based on rutile includes: collecting metallogenic porphyry samples from a survey region, and acquiring sampling point data of each sample; treating each metallogenic porphyry sample, and determining characteristic data of rutile in each metallogenic porphyry sample; according to a zirconium content of rutile, calculating a metallogenic temperature value of each metallogenic porphyry sample; testing an average titanium isotope value of each metallogenic porphyry sample; and determining the migration path of the ore-forming hydrothermal fluid according to sampling point coordinate data, temperature values, and titanium isotope values. A migration path of an ore-forming hydrothermal fluid is determined according to a temperature value determined based on a zirconium content of rutile and a titanium isotope value, which allows the accurate prediction of migration paths of ore-forming hydrothermal fluids for all porphyry copper deposits.

### CROSS-REFERENCE TO RELATED APPLICATION

This application is claims priority to Chinese Patent Application No. 202411492391.3 with a filing date of Oct. 24, 2024. The content of the aforementioned application, including any intervening amendments thereto, is incorporated herein by reference.

### TECHNICAL FIELD

The present application relates to the field of exploration geology, and in particular to a method for determining a migration path of an ore-forming hydrothermal fluid based on rutile.

### BACKGROUND

Porphyry deposits have characteristics such as large scale, shallow bury of ore bodies, and easy mining of ores. Porphyry deposits account for about 70% of the copper production worldwide, 95% or more of the molybdenum production worldwide, and 20% or more of the gold production worldwide. Therefore, porphyry deposits are one of the deposit types highly valued in the industry. For porphyry deposits, the enrichment and mineralization are allowed mainly through a magmatic hydrothermal fluid. The revealing of a migration direction of an ore-forming fluid and changes of a fluid composition and a mineral assemblage during a migration process is an effective way to lock the metal precipitation. A range and location of an ore body can be quickly locked by locking a migration path of an ore-forming hydrothermal fluid.

A fluid produced at an early stage of a porphyry mineralization system generally has characteristics such as high temperature and high oxygen fugacity, which is conducive to the migration of metals. However, at a metal precipitation stage, a temperature and an oxygen fugacity of the fluid decrease. The decrease of an oxygen fugacity is related to the presence of a large quantity of magnetite crystals during a cooling process. Therefore, a migration path of an ore-forming hydrothermal fluid of a porphyry deposit needs to be limited through the combination of changes in a temperature and an oxygen fugacity of a fluid.

A fluid inclusion is an effective manner to limit a temperature change of a fluid, but it is difficult to limit an oxygen fugacity of a fluid by this manner. Currently, an oxygen fugacity of a fluid is usually roughly limited in combination with a mineral assemblage, which has a poor accuracy.

A geological method is as follows: an alteration zone and a mineral assemblage relationship are found out through geological mapping to determine a migration path of a hydrothermal fluid. A mineralogical method is as follows: a migration path of a hydrothermal fluid is determined according to a spatial change of mineral geochemistry of an altered mineral. A migration path of a hydrothermal fluid can also be identified according to spatial changes in characteristics such as wavelengths and absorption peaks of short-wave infrared spectra for clay minerals such as mica and kaolin that are caused by temperature changes.

Geological methods, such as geological mapping, are time-consuming and expensive, require a lot of manpower and material resources, require technical personnel to have high professional knowledge, and are difficult to quantitatively determine a migration path of a hydrothermal fluid. Mineralogical and spectroscopic methods are mainly based on spatial changes in chemical and spectral characteristics such as wavelengths and absorption peaks of altered minerals such as chlorite and epidote that are caused by temperature changes. These methods have specified application prospects in the determination of a migration path of a hydrothermal fluid, but are not suitable for all porphyry copper deposits. Altered minerals such as chlorite and epidote are rarely developed in a potassic zone close to a hydrothermal fluid center for most of porphyry copper deposits, and there is hydrothermal fluid superimposition among different hydrothermal fluid centers for multi-hydrothermal center deposits. Thus, the chemical and spectral characteristics of altered minerals lose their spatial regularities, and it is impossible to accurately determine a migration path of a hydrothermal fluid. Therefore, there is a lack of a reliable method for determining a migration path of a hydrothermal fluid of a porphyry copper deposit.

### SUMMARY

An objective of the present disclosure is to provide a method for determining a migration path of an ore-forming hydrothermal fluid of a porphyry deposit based on rutile, which can accurately determine a migration path of an ore-forming hydrothermal fluid.

A method for determining a migration path of an ore-forming hydrothermal fluid based on rutile is provided, including the following steps:
S1, collecting metallogenic porphyry samples from a survey region, pre-treating each sample, and marking a location of each rutile mineral in each sample;
S2, measuring a zirconium content of each rutile mineral in each sample to determine an average zirconium content in each sample, and calculating a metallogenic temperature of each sample according to the average zirconium content;
S3, acquiring an average titanium isotope value of each sample; and
S4, determining the migration path of the ore-forming hydrothermal fluid according to coordinate data, the metallogenic temperature, and the average titanium isotope value of each sample.

Further, the S1 is specifically as follows: collecting the metallogenic porphyry samples spatially according to a density of exploration grid, and recording sampling coordinate data of a sampling point for each sample; and
grinding each sample into a polished section, and observing and recording a production status, a size, and a location of each rutile mineral in each sample. Further, the S2 is specifically as follows:
conducting in-situ micro-area element analysis for each rutile mineral in each sample to determine whether there is a uniform Zr content in rutile minerals, excluding a rutile mineral with a Zr-rich inclusion, retaining a rutile mineral without the Zr-rich inclusion, and measuring a zirconium content in the rutile mineral without the Zr-rich inclusion; for a sample including a plurality of rutile minerals, averaging zirconium contents in the plurality of rutile minerals to obtain an average zirconium content of the sample; and calculating a metallogenic temperature of the sample according to the average zirconium content of the sample.

Further, an equation for calculating a metallogenic temperature T of a sample is as follows:

$$T = 84.31 / ( 0.1428 - 0.0083144 \times {ln}^{{Zr}_{ppm}} ) . \qquad ( equation 1 )$$

Further, the S3 is specifically as follows:
testing an in-situ titanium isotope value of each rutile mineral in each sample; and for a sample including a plurality of rutile minerals, averaging in-situ titanium isotope values of the plurality of rutile minerals tested to obtain an average titanium isotope value of the sample.

Further, the S4 is specifically as follows:
analyzing whether there is any spatial change in the metallogenic temperature of each sample; if there is no spatial change, indicating that there is no hydrothermal fluid migration channel in the survey region; and if there is a spatial change:
linearly fitting metallogenic temperatures and average titanium isotope values of a plurality of samples, where if the metallogenic temperatures are negatively correlated with the average titanium isotope values, it indicates that the survey region has a metallogenic potential and an evolution direction from a high temperature to a low temperature among the metallogenic temperatures of the plurality of samples is a direction of the migration path of the ore-forming hydrothermal fluid; and
if the metallogenic temperatures are not correlated with or are positively correlated with the average titanium isotope values, it indicates that the survey region does not have a metallogenic potential and also does not have a hydrothermal fluid migration channel.

The technical solutions provided by the present disclosure have the following beneficial effects:

In the present disclosure, a spatial temperature distribution is calculated based on a zirconium content of rutile in a metallogenic porphyry sample, and whether an ore-forming hydrothermal fluid undergoes an oxygen fugacity reduction is determined by testing a titanium isotope value of a titanium isotope in rutile, so as to determine a migration path of an ore-forming hydrothermal fluid for a porphyry deposit. The present disclosure overcomes the problems such as complexity and inefficiency in geological and geochemical methods.

### BRIEF DESCRIPTION OF THE DRAWING

The present application will be further described below in conjunction with the accompanying drawing and embodiments.

FIG. 1 is a flow chart showing a method for determining a migration path of an ore-forming hydrothermal fluid based on rutile according to an embodiment of the present disclosure; and

FIG. 2 shows the correlation between a temperature and a titanium isotope in rutile during the method for determining a migration path of an ore-forming hydrothermal fluid of a porphyry deposit based on rutile in Example 1 of the present disclosure.

### DETAILED DESCRIPTION OF THE EMBODIMENTS

In order to make the technical features, objective, and effect of the present application clearly understood, the specific embodiments of the present application are described in detail with reference to the accompanying drawing.

Rutile minerals are extremely stable, and have low element diffusion rates. In addition, trace elements in rutile minerals are mainly controlled by contents and partition coefficients of trace elements in a molten fluid. Therefore, it is possible to identify a migration path of an ore-forming hydrothermal fluid of a porphyry deposit based on rutile. Rutile is an accessory mineral most widely distributed in porphyry deposits. Rutile is widely developed in potassic zones, sericitic zones, argillic zones, and propylitic zones of porphyry copper deposits. Titanium-rich minerals such as biotite and sphene undergo alteration during the interaction between an ore-forming fluid and a rock to finally produce hydrothermal rutile, thereby forming Rutile. Rutile can hardly be transformed by hydrothermal alteration at a later stage after formation. Therefore, rutile is an ideal mineral for investigating a hydrothermal fluid migration path of a porphyry mineralization system.

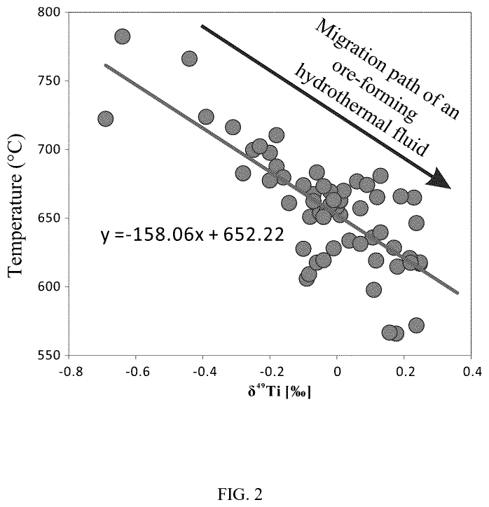

A temperature of a porphyry copper deposit decreases spatially from a center to a periphery of a hydrothermal fluid. Previous experimental petrological studies have shown that, when a pressure is close to 1 kbar, the zirconium and temperature in rutile meet the following relationship: T=84.31/(0.1428-0.0083144×ln^{Zr}^{ₚₚₘ}). As shown in FIG. 2, a change of a zirconium content in rutile can be used to quickly lock a migration direction of a hydrothermal fluid spatially, so as to determine a migration path.

However, it is necessary to further determine whether the hydrothermal fluid has a metallogenic potential. The reduction of an oxygen fugacity during evolution of a hydrothermal fluid is an effective indicator to determine whether the hydrothermal fluid has a metallogenic potential. The reduction of an oxygen fugacity during evolution of a fluid is due to the crystallization of fluid magnetite, and the crystallization of fluid magnetite will lead to the increase of titanium isotopes in the fluid. Therefore, a change of titanium isotopes in a fluid can be a prominent indicator to reflect whether the fluid has a metallogenic potential. 99% of crystalline rutile in a fluid is titanium, and thus rutile is an effective mineral for measuring titanium isotopes in the fluid. Therefore, a spatial temperature distribution can be calculated by testing zirconium contents of rutile minerals at different spatial locations, and then whether an ore-forming hydrothermal fluid undergoes an oxygen fugacity reduction is determined by testing a change of titanium isotopes in rutile, so as to determine a metallogenic potential of the ore-forming hydrothermal fluid and a migration path of the ore-forming hydrothermal fluid for a porphyry deposit.

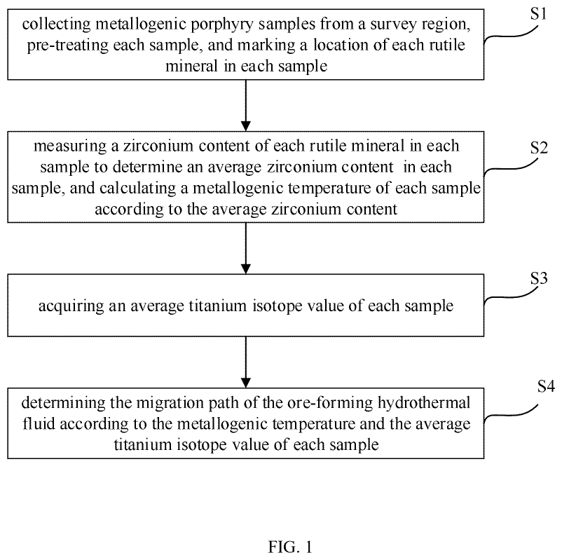

Therefore, as shown in FIG. 1, an embodiment of the present application provides a method for determining a migration path of an ore-forming hydrothermal fluid based on rutile, including:
S1: Metallogenic porphyry samples are collected from a survey region, each sample is pre-treated, and a location of each rutile mineral in each sample is marked.

### Specifically:

The metallogenic porphyry samples are collected spatially according to a density of exploration grid, and sampling coordinate data of a sampling point for each sample is recorded.

Each sample is grinded into a polished section, and a production status, a size, and a location of each rutile mineral in each sample are observed and recorded under a microscope.
S2: A zirconium (Zr) content of each rutile mineral in each sample is measured to determine an average zirconium content Zrₚₚₘ in each sample, and a metallogenic temperature of each sample is calculated according to the average zirconium content Zrₚₚₘ. A titanium dioxide content of each rutile mineral in each sample can also be obtained, and an average titanium dioxide content in each sample is obtained similarly.

### Specifically:

In-situ micro-area element analysis is conducted by laser ablation-inductively coupled plasma-mass spectrometry (LA-ICP-MS) for each rutile mineral in each sample to determine whether there is a uniform Zr content in rutile minerals, a rutile mineral with a Zr-rich inclusion is excluded, a rutile mineral without the Zr-rich inclusion is retained, and a zirconium content in the rutile mineral without the Zr-rich inclusion is measured. Because each sample includes a plurality of rutile minerals, zirconium contents in the plurality of rutile minerals are averaged to obtain an average zirconium content Zrₚₚₘ of each sample. It should be noted that the determination of whether there are Zr-rich inclusions in rutile according to LA-ICP-MS in the present disclosure is the prior art, and a specific detection process is not repeated in the present disclosure.

According to an equation 1, a metallogenic temperature T of a sample is calculated based on an average zirconium content Zrₚₚₘ of the sample:

$$T = 84.31 / ( 0.1428 - 0.0083144 \times {ln}^{{Zr}_{ppm}} ) . \qquad ( equation 1 )$$

S3: An average titanium isotope value of each sample is acquired.

### Specifically:

An in-situ titanium isotope value of each rutile mineral in each sample is tested through a combination of femtosecond laser ablation (fs-LA) and multi-collector inductively coupled plasma-mass spectrometry (MC-ICP-MS). Because each sample includes a plurality of rutile minerals, in-situ titanium isotope values of the plurality of rutile minerals tested are averaged to obtain an average titanium isotope value of each sample.
S4: The migration path of the ore-forming hydrothermal fluid is determined according to coordinate data, the metallogenic temperature, and the average titanium isotope value of each sample.

### Specifically:

A cooling direction of an ore-forming hydrothermal fluid is determined based on an evolution direction from a high temperature to a low temperature among metallogenic temperatures of a plurality of samples. If there is no spatial change in the metallogenic temperatures of the plurality of samples, it indicates that there is no hydrothermal fluid migration channel in the survey region.

The metallogenic temperatures and average titanium isotope values of the plurality of samples are linearly fitted. If the metallogenic temperatures are negatively correlated with the average titanium isotope values, it indicates that there are magnetite crystallization and oxygen fugacity reduction, and this series is the migration path of the ore-forming hydrothermal fluid. An evolution direction from a high temperature to a low temperature among the metallogenic temperatures of the plurality of samples is a direction of the migration path of the ore-forming hydrothermal fluid. If the metallogenic temperatures are not correlated with or are positively correlated with the average titanium isotope values, it indicates that the survey region does not have a metallogenic hydrothermal evolution and a metallogenic potential.

### Example 1

Step 1: Data acquisition and sample collection for a survey region and surrounding regions thereof

A porphyry copper deposit in the Kailas metallogenic belt was selected as the survey region. 60 metallogenic porphyry samples were collected spatially according to the density of exploration grid 100 m×100 m, and a length, a width, and a height of each sample were about 3 cm, 5 cm, and 8 cm, respectively. For each sampling point, coordinate data, field photos, detailed field records, and data such as lithologic, alteration, and mineralization characteristics of each sample needed to be acquired.
Step 2: Sample pretreatment.

Each sample was grinded into a polished section, and characteristics of each rutile mineral in each sample were observed under a microscope. A production status, a size, and a location of each rutile mineral in each sample were recorded in detail.
Step 3: Calculation of a metallogenic temperature value of each sample

In-situ micro-area element analysis is conducted by LA-ICP-MS for each rutile mineral in each sample. 10 or more rutile particles were analyzed for each sample. A titanium dioxide content and a zirconium content in each rutile mineral were recorded, and an average zirconium content Zrₚₚₘ and an average titanium dioxide content of each sample were calculated.

According to a signal curve of LA-ICP-MS, whether there is a uniform zirconium content in rutile minerals was determined, a rutile mineral with a zirconium-rich inclusion was excluded, and finally a rutile mineral without a zirconium-rich inclusion was retained. Then an average zirconium content Zrₚₚₘ of a sample was calculated. The average zirconium content Zrₚₚₘ of each metallogenic porphyry sample was substituted into an equation T=84.31/(0.1428-0.0083144×ln^{Zr}^{ₚₚₘ}) to calculate a metallogenic temperature value of each metallogenic porphyry sample.
Step 4: Testing of an average titanium isotope value of each sample

An in-situ titanium isotope value of each sample was tested through the combination of fs-LA and MC-ICP-MS.

The conventional data acquisition included 120 cycles, with 0.524 s set for each cycle. The first 25 cycles were responsible for monitoring the instrument background, and the subsequent 95 cycles were responsible for collecting sample signals. An actual titanium isotope ratio was calculated based on signal values of stable 50 to 60 cycles. Laser ablation parameters were 1 Hz, 20 μm, and 3.2 Jcm⁻². An average titanium isotope (δ⁴⁹Ti) value of each metallogenic porphyry sample was calculated.
Step 5: Determination of the migration path of the ore-forming hydrothermal fluid

According to coordinates, a metallogenic temperature value, and an average titanium isotope value of each sample, the migration path of the ore-forming hydrothermal fluid was determined. Test data information of each sample was shown in Table 1, and a linear fitting result between the metallogenic temperature value and the average titanium isotope value of each sample was shown in FIG. 2. As shown in FIG. 2, the metallogenic temperature values are negatively correlated with the average titanium isotope values of the plurality of samples in this survey region, indicating that this survey region undergoes magnetite crystallization and oxygen fugacity reduction and has a metallogenic potential, and an evolution direction from a high temperature to a low temperature among the metallogenic temperatures of the plurality of samples is a direction of the migration path of the ore-forming hydrothermal fluid of this survey region. Each spatial location on the migration path is depicted according to coordinates of each sample.

TABLE 1
Test results

| | | TiO₂ | | Temperature |
|---|---|---|---|---|
| Sample | δ⁴⁹Ti | (wt %) | Zr (ppm) | (° C.) |
| 115-01 | −0.18 | 95.31 | 653 | 710 |
| 115-02 | −0.08 | 92.03 | 333 | 651 |
| 115-03 | −0.31 | 92.66 | 694 | 716 |
| 115-04 | −0.10 | 94.78 | 250 | 628 |
| 115-05 | −0.09 | 92.13 | 188 | 606 |
| 115-06 | −0.28 | 96.17 | 481 | 683 |
| 115-07 | −0.64 | 96.74 | 1331 | 782 |
| 115-08 | −0.39 | 97.01 | 751 | 724 |
| 115-09 | −0.25 | 93.10 | 582 | 700 |
| 115-10 | −0.69 | 92.19 | 740 | 722 |
| 115-11 | −0.44 | 96.71 | 1144 | 766 |
| 115-12 | −0.14 | 96.94 | 375 | 661 |
| 115-13 | −0.08 | 96.10 | 196 | 609 |
| 115-14 | 0.12 | 96.70 | 224 | 619 |
| 115-15 | 0.18 | 96.31 | 107 | 566 |
| 115-16 | 0.24 | 95.89 | 315 | 646 |
| 115-17 | 0.25 | 89.84 | 217 | 617 |
| 115-18 | 0.11 | 95.68 | 277 | 636 |
| 115-19 | 0.16 | 95.86 | 109 | 567 |
| 115-20 | 0.24 | 96.34 | 117 | 572 |
| 115-21 | 0.25 | 96.51 | 219 | 618 |
| 115-22 | 0.22 | 94.39 | 229 | 621 |
| 115-23 | 0.04 | 95.98 | 269 | 634 |
| 115-24 | −0.18 | 94.75 | 509 | 688 |
| 115-25 | −0.16 | 94.47 | 465 | 680 |
| 115-26 | −0.20 | 94.17 | 454 | 677 |
| 115-27 | −0.07 | 93.58 | 405 | 667 |
| 115-28 | −0.06 | 92.21 | 219 | 618 |
| 115-29 | −0.05 | 95.95 | 344 | 654 |
| 115-30 | −0.07 | 96.56 | 381 | 662 |
| 115-31 | 0.01 | 95.49 | 338 | 652 |
| 115-32 | 0.00 | 96.03 | 363 | 658 |
| 115-33 | −0.03 | 96.01 | 350 | 655 |
| 115-34 | −0.06 | 95.54 | 485 | 683 |
| 115-35 | 0.01 | 96.05 | 383 | 663 |
| 115-36 | −0.04 | 96.69 | 224 | 619 |
| 115-37 | 0.06 | 95.03 | 451 | 677 |
| 115-38 | 0.01 | 96.41 | 384 | 663 |
| 115-39 | −0.02 | 96.57 | 369 | 660 |
| 115-40 | 0.02 | 96.60 | 417 | 670 |
| 115-41 | −0.20 | 97.64 | 569 | 698 |
| 115-42 | 0.09 | 95.31 | 437 | 674 |
| 115-43 | −0.23 | 91.54 | 598 | 702 |
| 115-44 | −0.04 | 96.99 | 332 | 651 |
| 115-45 | −0.01 | 90.31 | 251 | 628 |
| 115-46 | 0.23 | 89.67 | 393 | 665 |
| 115-47 | 0.11 | 94.01 | 169 | 598 |
| 115-48 | 0.13 | 95.42 | 472 | 681 |
| 115-49 | 0.12 | 92.29 | 395 | 665 |
| 115-50 | −0.02 | 97.38 | 414 | 669 |
| 115-51 | 0.18 | 89.74 | 211 | 615 |
| 115-52 | 0.13 | 96.56 | 290 | 640 |
| 115-53 | 0.17 | 96.60 | 252 | 629 |
| 115-54 | −0.01 | 97.32 | 385 | 663 |
| 115-55 | 0.22 | 94.37 | 219 | 618 |
| 115-56 | −0.10 | 97.36 | 436 | 674 |
| 115-57 | 0.19 | 93.32 | 397 | 666 |
| 115-58 | 0.07 | 98.02 | 358 | 657 |
| 115-59 | 0.07 | 96.16 | 261 | 631 |
| 115-60 | −0.04 | 96.00 | 432 | 673 |

In the present disclosure, rock samples are collected in a spatial system, trace elements and titanium isotopes of rutile at different spatial locations are analyzed by LA-ICP-MS, a formation temperature of rutile at each location is calculated, and finally a migration direction of an ore-forming hydrothermal fluid is accurately determined according to changes in temperatures and titanium isotopes. The method of the present disclosure can well explore the prospecting breakthrough of a porphyry mineralization system, and is a novel economical and efficient method for mineral exploration. The method of the present disclosure can save 90% or more of money and 80% or more of time compared with the traditional exploration methods.

The above is only an exemplary embodiment of the present disclosure, and cannot limit the scope of the present disclosure. Any equivalent changes and modifications made in accordance with the teachings of the present disclosure still fall within the scope of the present disclosure. Those skilled in the art can easily think of other embodiments of the present disclosure after considering the disclosure of the specification and practical truth.

The present application is intended to cover any variations, purposes, or adaptive changes of the present disclosure. Such variations, purposes, or applicable changes follow the general principle of the present disclosure and include common knowledge or conventional technical means in the technical field which is not mentioned in the present disclosure. The specification and embodiments are considered as merely illustrative, and the scope and spirit of the present disclosure are defined by the claims.

What is claimed is:

1. A method for determining a migration path of an ore-forming hydrothermal fluid based on rutile, comprising the following steps:
S1, collecting metallogenic porphyry samples from a survey region, pre-treating each sample, and marking a location of each rutile mineral in each sample;
S2, measuring a zirconium content of each rutile mineral in each sample to determine an average zirconium content Zrₚₚₘ in each sample, and calculating a metallogenic temperature of each sample according to the average zirconium content Zrₚₚₘ;
S3, acquiring an average titanium isotope value of each sample; and
S4, determining the migration path of the ore-forming hydrothermal fluid according to the metallogenic temperature and the average titanium isotope value of each sample; wherein the step S4 is specifically as follows:
forming a spatial distribution of metallogenic temperatures of the metalogenic porphyry samples in the survey region according to the metallogenic temperature of each sample; determining whether there is any change in the metallogenic temperatures of the metalogenic porphyry samples in the survey region; when there is no change, indicating that there is no hydrothermal fluid migration channel in the survey region; and when there is a spatial change:
linearly fitting the metallogenic temperatures and average titanium isotope values of the metallogenic porphyry samples, wherein when the metallogenic temperatures are negatively correlated with the average titanium isotope values, determining that the survey region has a metallogenic potential and an evolution direction from a high temperature to a low temperature among the metallogenic temperatures of the metallogenic porphyry samples is a direction of the migration path of the ore-forming hydrothermal fluid; and
when the metallogenic temperatures are not correlated with or are positively correlated with the average titanium isotope values, determining that the survey region does not have a metallogenic potential and also does not have a hydrothermal fluid migration channel.

2. The method according to claim 1, wherein the step S1 is specifically as follows:
collecting the metallogenic porphyry samples spatially according to a density of exploration grid, and recording sampling coordinate data of a sampling point for each sample; and
grinding each sample into a polished section, and observing and recording a production status, a size, and a location of each rutile mineral in each sample.

3. The method according to claim 1, wherein the step S2 is specifically as follows:
conducting in-situ micro-area element analysis for each rutile mineral in each sample to determine whether there is a uniform Zr content in rutile minerals, excluding a rutile mineral with a Zr-rich inclusion, retaining a rutile mineral without the Zr-rich inclusion, and measuring a zirconium content in the rutile mineral without the Zr-rich inclusion; for a sample comprising a plurality of rutile minerals, averaging zirconium contents in the plurality of rutile minerals to obtain an average zirconium content Zrₚₚₘ of the sample; and calculating the metallogenic temperature of the sample according to the average zirconium content Zrₚₚₘ of the sample.

4. The method according to claim 3, wherein an equation for calculating the metallogenic temperature T of the sample is as follows:
$$T = 84.31 / ( 0.1428 - 0.00831 \; 44 \times {ln}^{{Zr}_{ppm}} ) .$$

5. The method according to claim 1, wherein the step S3 is specifically as follows:
testing an in-situ titanium isotope value of each rutile mineral in each sample; and for a sample comprising a plurality of rutile minerals, averaging in-situ titanium isotope values of the plurality of rutile minerals tested to obtain an average titanium isotope value of the sample.
